(19) United States
(12) Patent Application Publication
(10) Pub. No.: US 2006/0106277 A1
(43) Pub. Date: May 18, 2006

(54) System for the treatment of stress urinary incontinenc

(76) Inventor: Jesus Romero Maroto, El Campello (ES)

Correspondence Address: SUGHRUE MION, PLLC, 2100 PENNSYLVANIA AVENUE, N.W., SUITE 800, WASHINGTON, DC, 20037, US

(21) Appl. No.: 10/515,770

(22) PCT Filed: Mar. 18, 2003
(86) PCT No.: PCT/ES03/00128

§ 371 Date: Nov. 24, 2004

(30) Foreign Application Priority Data
May 29, 2002 (ES) P200201227

(51) Int. Cl. A61F 2/02

(52) U.S. Cl. 600/37; 606/151; 606/185; 623/23.66

(57) ABSTRACT

The invention relates to an improved system for the treatment of incontinence. The inventive system comprises means of returning the vesical neck and the urethra to the original position thereof in a less aggressive manner, through the use of a mesh that can be positioned beneath the urethra and tautened to a desired value. Said system comprises: a mesh positioning element in the form of a tool consisting of a handle and a blunt needle, said needle having a wide through hole disposed at the distal end thereof; and a mesh element with a central urethral portion, both ends thereof terminating in filiform elements. The aforementioned mesh comprises different sets of through holes which are disposed at both ends thereof and on either side of the intermediate urethral portion.

### OBJECT OF THE INVENTION

The present invention relates to an improved system for the treatment of stress urinary incontinence, which provides essential characteristics of novelty and appreciable advantages in relation to the known means used for the same purposes in the current state of the art.

More particularly, the system proposed by the invention develops means especially indicated for the treatment of said pathology, which appreciably improves the application of the techniques used at present, especially that known as the sling technique, using for this elements which, being of a similar conception to conventional ones, have been advantageously modified and simplified. Said means include a single thread passer, of a smaller calibre than those currently known, provided with an orifice in its distal end which permits the holding and subsequent pulling of the mesh so that each one of its ends terminates in two threads which may be threaded through the eye of the needle, to perform the positioning of the mesh by pulling, said mesh also having the advantageous modification of having being provided with small holes which permit threads, intended to facilitate the pulling operation and that of tightening/loosening the mesh once positioned, to pass through.

The field of application of the invention is in the industrial sector dedicated to the manufacturing of utensils, instruments and devices for medical use, and in particular medical-surgical use.

### BACKGROUND AND SUMMARY OF THE INVENTION

The fact that stress urinary incontinence is a frequent pathology in women is generally known, consisting of urine loss when abdominal stress is performed such as coughing, going from the sitting position to that of standing up or walking.

Said problem constitutes an illness which frequently causes a serious reduction in the quality of life, as a consequence of the voluntary limitation of multiple activities, due to fear and shame that said loss may be noted by other people, and which often leads to situations of loneliness and isolation.

The anatomic alteration which lies behind this urine loss symptomology, consists of the descent of the vesical neck and the urethra, due to the alteration of the perineal muscles, caused, amongst other reasons, due to giving birth. This vesical neck and urethra descent means that abdominal pressures are not correctly transmitted thereto, this set of altered pressures being the cause of the involuntary urine leaks.

Until now, some techniques have been proposed which tend to provide solutions to the problem posed. In any case, the solution to said problem of incontinence is surgical and consists of returning the vesical neck and urethra to their original position, then providing them with a support which permits the correct transmission of abdominal pressures thereto. Amongst the techniques used, that known as sling is the one which provides a more efficient, lasting solution.

This sling technique consists of placing a tissue band, whether from the patient herself or heterologous tissue (marlex, silicone and, lately and with best results, prolene), in a sub-cervical or sub-urethral position, so that once said band is fixed in different points according to the technique, it lifts and supports the vesical neck and the urethra in their original position, thus enabling pressure transmission and avoiding urine loss.

The tissue band can be sufficiently long to go from the urethra to the hypogastrium, or it can consist only of a small patch joined to 4 threads, which are later attached to the abdominal wall.

The aggressiveness of the technique has been considerably reduced since it is performed intravaginally, without needing to open the pelvic cavity, and since prolene has been used, as it avoids having to collect aponeurosis from the same patient.

Recently, within the concept of this surgery as non-evasive, two sets have come onto the market for urinary incontinence surgery, identified by the acronyms TVT and SPARC. Both consist of a polypropylene mesh, surrounded by a plastic cover, and a connector system consisting of a needle with a handle. Both techniques are differentiated in that, in the case of TVT, the needle is passed through the vagina towards the hypogastrium, needing to separate the urethra with a probe and a catch, whilst in the case of the SPARC technique, this step is performed in the direction of hypogastrium to the vagina, without the urethra needing to be separated by a probe. In both cases, the polypropylene mesh used, surrounded by a plastic cover, is passed through the vagina to the hypogastrium, which has facilitated the mesh passing through, leaving the latter positioned subcutaneously, where it is fixed due to the properties it has been provided with.

In both techniques, and all those wherein the sling system is used, the most important problem arising consists of the tension given to the sling being correctly adjusted, being a special feature that conditions the success or failure of this type of surgery. Furthermore, if it is subjected to excessive tension, a urinary obstruction may occur, so that the patient will have difficulties in eliminating urine. In contrast, if sufficient tension is not provided, the patient will then continue to have an incontinence problem.

There is, therefore, a practical difficulty associated with the fact that there is no way of calculating the most appropriate tension, having to do so randomly and approximately. Once the operation has concluded, it is then not possible to correct the excess or lack of tension, which leads to a failure rate estimated at between 10% and 20% of the operations. Furthermore, this problem is the cause of all failures which occur with the application of this technique.

The existence of a system identified as REEMEX is also known in the market, wherein a subcutaneous implant of a device in the form of a pulley is performed in the hypogastrium, which permits, with the use of a screwdriver, and during the postoperative, to increase the tension given to a sling formed by a small patch of tissue fastened with four threads. This system is expensive, complex and suffers from considerable potential complications, due to the fact that it requires the implantation of a foreign body. It also can be loosened if the sling has been made with threads, but the system cannot be used when it uses a sling only formed from mesh, i.e. of TVT or SPARC type.

Finally, there is another system currently in existence, although there is no knowledge of its commercialization, consisting of a development by Dr. Gil Vernet, which includes a liquid reservoir implanted in subcutaneous tissue in the hypogastrium. The threads which fasten the sling have been attached to said reservoir. The system allows, if there is little tension, that the reservoir can be filled with liquid, thereby increasing the tension. The system is of equally difficult clinical application, and cannot be used with complete meshes.

In accordance with the previous description, the systems used in the application of the sling technique have a series of drawbacks associated with their use, which do not permit the fully satisfactory use thereof.

The present invention has proposed the main objective of developing a system whereby an efficient, quick, economic solution is provided to said problems of the current art, especially that regarding the application of the sling system.

This objective has been fully attained by the system of the invention, wherein a mesh positioning device (or thread passer) and a mesh, intervene as fundamental elements.

As regards the thread passer, it has the advantage that it is of smaller calibre than those currently used, meaning it is less aggressive, with a considerable reduction in the possibilities of causing iatrogenic lesions; furthermore, the thread passer is universal, i.e. it serves for both directions, being able to pass from vagina to hypogastrium, or from hypogastrium to vagina; also, regarding the connection system of the mesh to thread passer, it has been considerably simplified, as a hole in the distal part of the needle permits threading the end part of the mesh formed by threads.

As regards the mesh provided for the invention for its application with a view to maintaining the vesical neck and the urethra in an optimum position, it has certain advantageous characteristics in comparison with those used at present, amongst which we can be state, for example, the fact that it has no additional cover requires no withdrawal or extraction period as occurs at present, thus simplifying the procedure; furthermore, the mesh terminates in a reduction at the ends to two threads, which are those which are threaded in the thread passer and achieve the positioning of the mesh by pulling; finally, it should also be stated, as a particularly important characteristic, the fact that the mesh has holes distributed thereon, in predetermined positions, once the mesh has been positioned, on a aponeurosis of the rectus level and periurethral level, allowing threads to be introduced in said holes, whose ends are positioned, once the operation has concluded, outside the lines of vaginal and retropubic suture, so that, acting on the vaginal or retropubic threads, it can be tightened/loosened with a view to eliminating any obstruction or, respectively, correcting the incontinence, if it persisted, being something that can be performed the day after the operation, with the patient awake, carrying out her typical activities, thus allowing any dysfunction caused from the sling implantation to be corrected.

### BRIEF DESCRIPTION OF THE DRAWINGS

These and other characteristics and advantages of the invention will be more clearly shown from the detailed description below of a preferred embodiment, given only by way of illustrative and non-limitative example, with reference to the attached drawing, wherein

FIG. 1 shows side elevation and front elevation views of the mesh positioning element according to the invention, and

FIG. 2 is an illustrative representation of two alternative meshes, configured in accordance with the requirements of the system of the invention.

### DESCRIPTION OF A PREFERRED EMBODIMENT

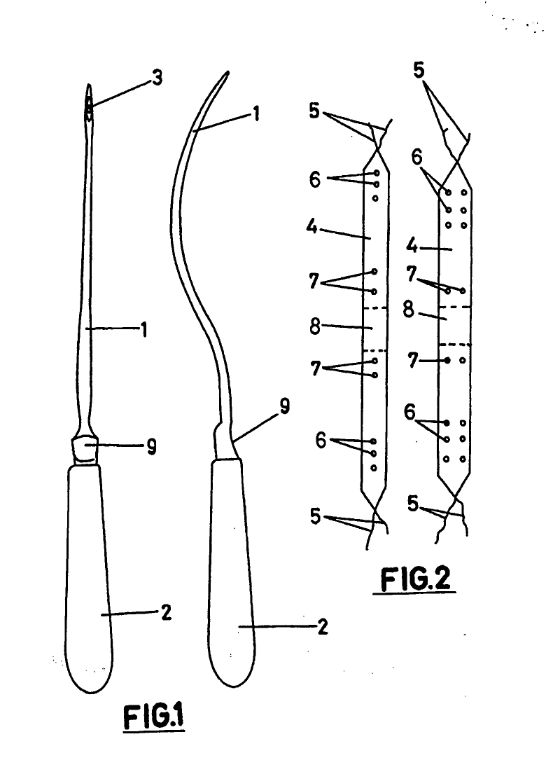

As stated above, the detailed description of the preferred embodiment of the invention will be made using the attached drawings, whereby numerical references will be made to designate the system components and the parts which define each one thereof. Thus, dealing firstly with the representation of FIG. 1, we can observe different views corresponding to the side elevation and front elevation views of a mesh positioning device (or thread passer), configured so that therein we can distinguish a needle portion (1) and a handle portion (2). The needle portion (1), is elongated, with a predetermined length sufficient for the purpose for which it is intended, and it has been provided with a certain curve throughout, terminating in a blunt point at the distal end thereof. A through hole (3) has been provided in the proximity of this distal end, which is slightly elongated, coinciding with the longitudinal direction of the needle, and of considerable size, which can especially be observed in the front view of the positioning device. As will be understood, the positioning device can be constructed with the use of very varied materials which allow correct sterilization, said materials including both those of perishable and disposable type and reusable type.

Furthermore, the handle portion (2) is represented with a conventional form, ergonomically shaped to permit easy, comfortable gripping by the device user, provided with a depression (9) to support the thumb with a view to safer gripping, and of dimensions which facilitate the unit's handling.

FIG. 2 shows the schematic representation of a portion of mesh of the type used as support in the correct positioning of the vesical neck and urethra. Said mesh portion is indicated with number (4), and consists of a band which, in its preferred embodiment, has an approximately rectangular shape, whose ends are narrowed and converge to terminate in filiform elements (5) at each one of its ends, whilst, on areas positioned around each end, and, where appropriate, in other intermediate areas, said mesh portion has been provided with several respective holes (6, 7).

In accordance with both alternatives of embodiment of the mesh, the holes (6, 7) can be grouped in different forms, longitudinally or transversally, in rows and/or lines, depending on the number of holes made in accordance with the mesh width. This special feature can be easily observed in the examples illustrated in FIG. 2, wherein, in the first alternative or mesh with less width, there are sets of three holes (6) beside the ends arranged longitudinally in rows, and sets of two holes (7) at both sides of an intermediate area (8), or urethral area, whilst in the second alternative or mesh with greatest width, we can observe sets of six holes beside both ends, arranged in two parallel rows of three holes each, whilst on both sides of the urethral area (8), two transversally aligned holes have been represented. Of course, as we have mentioned, the arrangement can be any other.

As will be understood, the inclusion of said holes (6, 7), permits the positioning device (1) to be applied with a view to pulling from any position, or even passing threads (not represented) through one or more of said holes, to be able to be then threaded through the hole (3) of the positioning device, and to be able to suitably adjust the positioning of the urethra which, in short, is the aim sought.

Preferably, two of the holes (7) of the mesh, will be arranged at approximately 1 and 2 cm from the urethral apposition area (8), whilst the other holes are arranged starting approximately five centimetres from the urethral area. The threads which pass through the holes (6), will be, once the operation has finished, positioned outside the vaginal and retropubic suture lines. As previously mentioned, by acting on the vaginal and retropubic lines, we can adjust the tension of the mesh to the most suitable tension for the patient's pathological characteristics. Furthermore, this form of mesh used by the system of the invention, also permits that the threads (5), projected from each end of the mesh (4), can be threaded in the hole (3) of the positioning device, so that it can be taken to its correct position.

As will be understood, the configuration adopted by the system components permit that, from the use of the latter, a series of advantages are derived, from which, those summarised below stand out as most important:

The procedure is less aggressive, by virtue of the needle used having a smaller calibre;

The product is less expensive, since plastic covers are eliminated, simplifying the joining of the mesh to the thread passer, with the same needle serving for both approach directions;

It is now possible to achieve what had not been attained up to now, and which consists of the modification of the tension given to the sling with the patient awake, therefore correcting any dysfunction (obstruction or incontinence) produced in the prior operation.

It is not considered necessary to expand on the content of this description for someone skilled in the art to understand its scope and the advantages derived from the invention, as well as to develop and put into practice the object thereof.

Nevertheless, it should be understood that the invention has been disclosed according to the preferred embodiment thereof, which means it can be modified without requiring any alterations of the essence of said invention, said modifications being able to affect, especially, the shape, size and/or manufacturing materials of the unit or any of the parts of the system, used.

1. Improved system for the treatment of stress urinary incontinence, which provides means to return the vesical neck and the urethra to its original, correct position with the use of a mesh which can be positioned beneath the urethra and tightened to a predetermined value corresponding to said desired position of the vesical neck and the urethra, which is characterized in that the system comprises:
A mesh positioning device (or thread passer), which has a handle portion (2), economically configured to be easily and comfortably gripped, and a needle (1) appreciably curved throughout, and which, at its distal end, terminates in a blunt point, and has, beside this end, a wide slightly elongated through hole (3), extended in accordance with the longitudinal direction of the needle (1), and
a mesh element (4) in the form of an elongated band, of an optionally variable width, wherein a urethral area (8) is defined in central position, and which narrows at both ends, terminating in filiform elements (5) adapted to be able to be threaded through the eye (3) of the needle (1) during the operations.

2. System according to claim 1, characterized in that said mesh (4) has been provided with several holes in different positions, in particular sets of holes (7) at each side of the urethral area (8) and other sets of holes (6) positioned throughout the mesh at each side of the urethral area and prepared to allow the passing of other threads through them, or even allow the introduction of the needle (1) point of the mesh positioning device during said mesh tightening operations.

3. System according to claim 2, characterized in that two of the holes (7) beside the urethral area are positioned at a distance of approximately 1 and 2 cm respectively thereto.
